(19) United States
(12) Patent Application Publication
(10) Pub. No.: US 2018/0008307 A1
(43) Pub. Date: Jan. 11, 2018

(54) TUBE INTRODUCERS AND ASSEMBLIES

(71) Applicant: SMITHS MEDICAL INTERNATIONAL LIMITED, Ashford (GB)

(72) Inventor: Stephen James Field, Canterbury (GB)

(73) Assignee: SMITHS MEDICAL INTERNATIONAL LIMITED, Ashford (GB)

(21) Appl. No.: 15/545,368

(22) PCT Filed: Jan. 19, 2016
(86) PCT No.: PCT/GB2016/000012

§ 371 Date: Jul. 21, 2017

(30) Foreign Application Priority Data
Feb. 6, 2015 (GB) 1502064.7

(51) Int. Cl. A61B 17/34; A61M 16/04; A61B 17/00

(52) U.S. Cl. CPC A61B 17/3415; A61M 16/0472; A61M 16/0429; A61B 2017/00314; A61B 2017/347

(57) ABSTRACT

An introducer for a tracheostomy tube is formed of several articulated sections (24) that can be changed from a relatively flexible state to a more rigid state by tightening a tension member extending along the introducer using an actuator at its machine end. In its flexible state the introducer can be inserted into and removed from the tube. The introducer is put in its more rigid state for use in inserting the tube into a tracheostomy. Alternatively or additionally the introducer could have several gripping rings on its outside towards its patient end and two elongate members that can be slid relative to one another to enlarge the patient end so that the gripping rings engage and grip the inside of the tube, thereby enabling the introducer to pull the tube through the tracheostomy from its patient end.

This invention relates to tube introducers of the kind for insertion within the bore of a medico-surgical tube.

In various medical and surgical procedures it is necessary to introduce a plastics tube into the body through a natural or surgically-created opening. It is often desirable for the tube to be flexible, in order to conform to the anatomy of a body passage or to reduce trauma to patient tissue. Flexible tubes are more difficult to insert by themselves so it is often necessary to support the tube internally by a stiffer introducer or obturator that can be removed once the tube has been inserted to the desired position. The introducer provides the maximum support for the tube if it is a tight fit within the tube, especially where the tube is highly flexible. Introducers that fit tightly, however, have a disadvantage in that they can be difficult to insert and remove, especially when the tube is located in the body. This is a particular problem if the tube has a contorted shape with small radius bends. Attempts to pull a tight-fitting introducer out of a tube can disturb the positioning of the tube and cause excessive forces on patient tissue in the region of the tube. One example of a procedure where introducers or obturators are used to support a tube is in the insertion of a tracheostomy tube where a tracheostomy tube is inserted through a surgically-made opening into the trachea so that the patient end of the tube locates in the trachea and its opposite, machine end projects outwardly from the surface of the neck. It is important in this procedure that the introducer provides sufficient support to enable the tube to be pushed through the opening between relatively stiff tracheal cartilages but it is also important that the introducer can be removed easily and quickly since air flow to and from the patient's respiratory passages will be prevented or substantially reduced until the introducer has been fully removed.

It is an object of the present invention to provide an alternative tube introducer.

According to one aspect of the present invention there is provided a tube introducer of the above-specified kind, characterised in that the introducer is selectively configurable from a first state where it is removable from the machine end of the tube to a second state where it supports the tube during insertion into the body.

The first state may be less rigid than the second state. The introducer may include a plurality of articulated sections that, in the first state, are only loosely retained with one another such that they can flex relative to one another and, in the second state, are retained more rigidly with one another. The introducer preferably includes a tension member extending along the introducer from its patient end to its machine end and an actuator at the machine end of the actuator arranged to apply tension to the tension member in the second state. Alternatively, in the second state the introducer may be expanded to grip the inside of the tube. The introducer may be expanded only in a region towards the patient end of the introducer. The introducer may have a plurality of gripping rings towards its patient end, the gripping rings being expanded in diameter in the second state. The introducer may have a tapering patient end adapted to project from the patient end of the tube within which it is inserted such as to help guide insertion of the tube into the body.

According to another aspect of the present invention there is provided a tube introducer adapted for insertion within the bore of a medico-surgical tube from the machine end of the tube, characterised in that the introducer has at least a region along its length towards its patient end with an external dimension that can be selectively changed from a first dimension where it enables the introducer to be slid into and out of the tube to a second, larger dimension where the outside of a part at least of the region can grip the inside of the tube to resist relative movement between the introducer and the tube.

According to a further aspect of the present invention there is provided a tube introducer having a patient end adapted for insertion in the bore of a medico-surgical tube to extend substantially to the patient end of the tube and a machine end adapted to project externally beyond the machine end of the tube, characterised in that the introducer is arranged selectively to grip the inside of the tube close to the patient end of the tube such that the tube and introducer can be inserted into a body cavity by pushing the machine end of the introducer so as to pull the tube into the body cavity from its patient end.

The introducer may include two elongate components movable relative to one another from a first position where the introducer can be slid into and out of a tube to a second position where a part at least of the introducer grips the inside of the tube. The two elongate components may include an outer sleeve and an inner elongate component that can be displaced longitudinally relative to the outer sleeve such that in the first position a part at least of the outer sleeve has a first diameter less than the internal diameter of the tube and in the second position the part of the outer sleeve has a larger diameter sufficient to grip the inside of the tube.

According to a fourth aspect of the present invention there is provided an assembly of a medico-surgical tube and a tube introducer according to the above one, other or further aspect of the present invention wherein the introducer is inserted within the tube with the patient end of the introducer protruding at the patient end of the tube and the machine end of the introducer accessible at the machine end of the tube.

The tube is preferably a tracheostomy tube.

Several different embodiments of introducer, according to the present invention, will now be described, by way of example, in which:

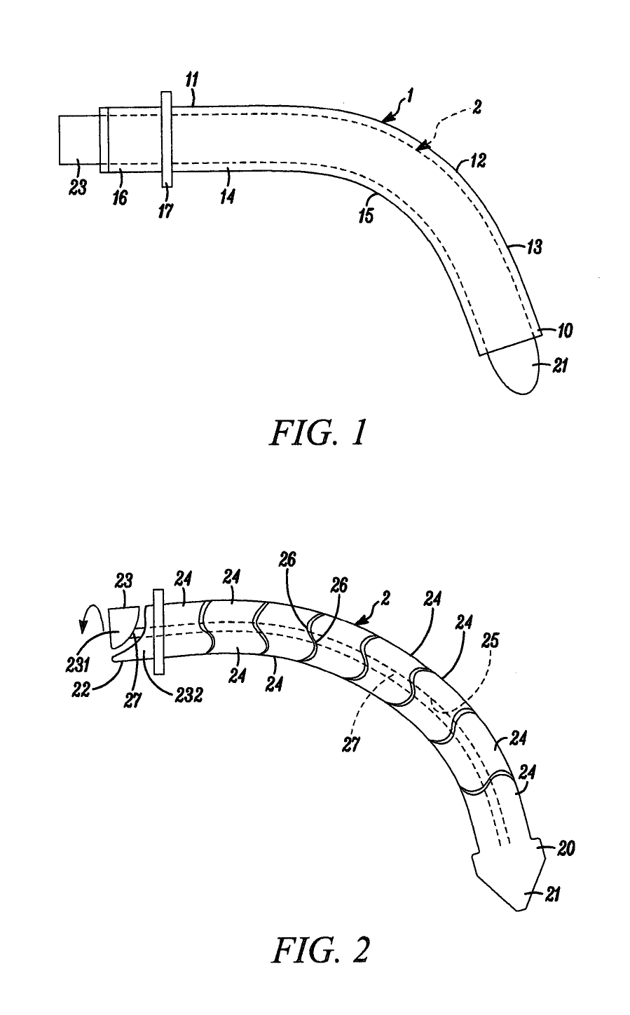

FIG. 1 is a side elevation view of an assembly of a tracheostomy tube and an introducer;

FIG. 2 is a side elevation view of a first form of introducer;

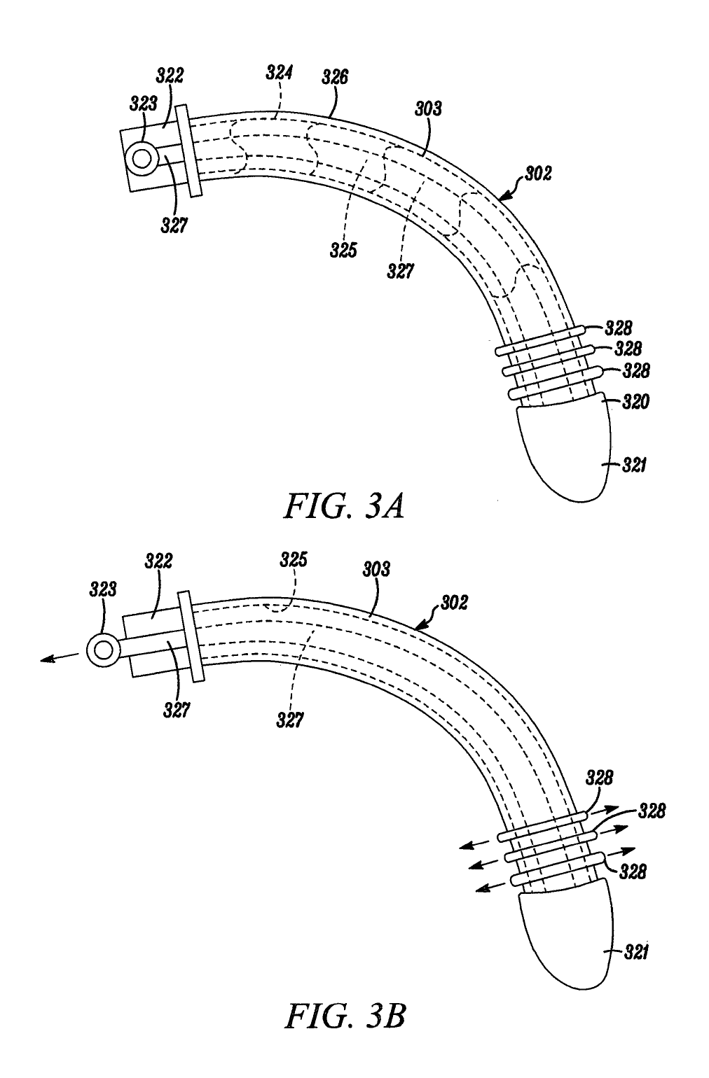

FIG. 3A is a side elevation view of a second form of introducer in its natural state where it is free to be inserted into and removed from a tracheostomy tube;

FIG. 3B is a side elevation view of the introducer of FIG. 3A in an expanded state where it grips the inside of the tracheostomy tube;

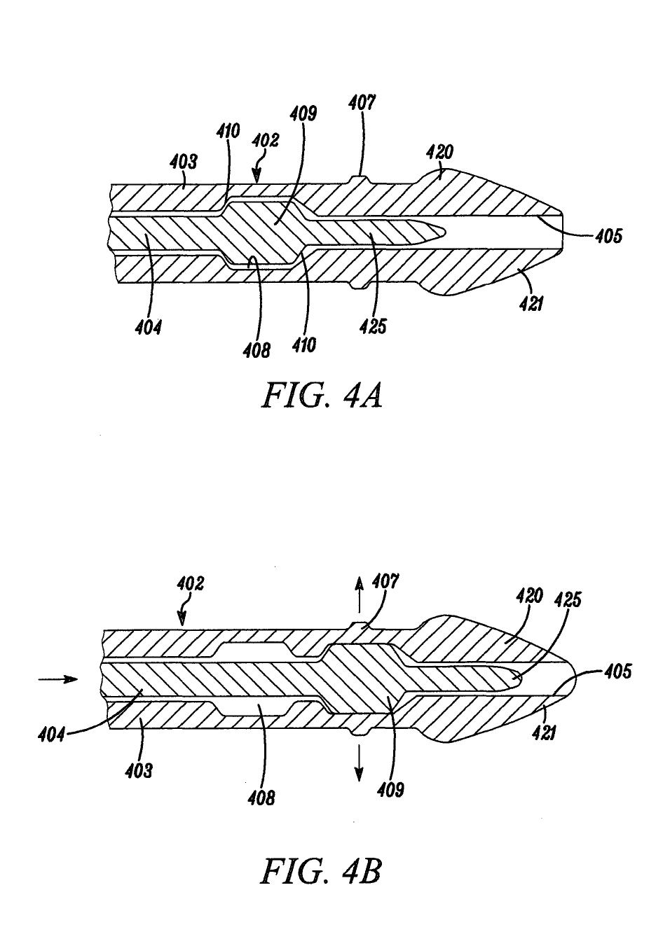

FIG. 4A is a side elevation view of the patient end of a third form of introducer in its natural state where it is free to be inserted into and removed from a tracheostomy tube; and

FIG. 4B is a side elevation view of the patient end of the introducer of FIG. 4A in an expanded state where it grips the inside of the tracheostomy tube.

With reference first to FIGS. 1 and 2 there is shown a tracheostomy tube and introducer assembly comprising a tracheostomy tube 1 and an introducer 2 inserted within the tube. The term “introducer” is used herein also to include any device inserted within a tube to help insertion of the tube into the body and includes obturators. The tracheostomy tube 1 may be of a conventional kind with a patient end 10 adapted to be located within the trachea and a machine end 11 adapted to project externally from the neck surface. The shaft 12 of the tube 1 may be of various shapes but, in this example, comprises a straight patient end portion 13, a straight machine end portion 14 and a curved intermediate portion 15. The machine end 11 of the tube 1 has a conventional 15 mm female connector 16 by which connection is made to the tube, and a flange 17 to which a neck strap (not shown) can be attached in order to secure the tube about the patient's neck. The tube 1 could be provided with a conventional inflatable sealing cuff close to its patient end but is shown as being without any such cuff.

The introducer 2 is shown more clearly in FIG. 2 and is adapted to be configurable from a first state in which it can be freely inserted into and removed from the tube to a second state where it supports the tube 1 while inserting the tube into the body. More particularly, in this first embodiment, the introducer 2 resembles in construction that of a “push puppet” toy that has a body or legs of separate sections and stands upright when tension is applied to hold the sections together but collapses when a button in the base of the toy is pressed to release the tension. The introducer 2 has a patient end 20 shaped to form a nose portion 21 that is pointed or rounded to help guide insertion of the assembly through the tracheostomy. When the introducer 2 is fully inserted in the tube 1 the nose portion 21 protrudes from the patient end 10 of the tube. At its machine end 22 the introducer 2 has an actuator 23 that protrudes rearwardly from the connector 16 of the tube 1 so as to be accessible by hand. Between the nose portion 20 and the actuator 23 the introducer 2 is divided into a plurality of separate articulated sections 24 of which eight are illustrated in this example. The sections 24 are of cylindrical or tubular shape having a bore 25 extending along their length and are formed with curved, profile ends 26 shaped to allow relative angular displacement between adjacent sections in at least one plane (being that of the plane of curvature of the tube 1). The introducer need not have a circular section but could, for example, have a cruciform or other shape that provides a channel between the introducer and the inside of the tube along which the patient can breathe during intubation.

An elongate tension member 27 extends along the length of the introducer 2, through the bore 25 of the sections 24 between the nose portion 20 and the actuator 23. The tension member 27 may take the form of a flexible rod, web, wire or the like and may be inelastic or have some resilience. The sections 24 may be linked mechanically together or retained together solely by the tension member 27. The actuator 23 may take many different forms and serves selectively to apply a tension force to the tension member 27 or to relax any such tension force. The actuator 23 illustrated has two cam members 231 and 232 one of which (attached with the tension member 27) can be rotated relative to the other (attached to the rear end section 24) to move it axially and thereby apply a pulling, tension force to the tension member 27. Other forms of actuator could include screw thread devices, hinged levers or a simple pull member with some form of lock to retain it in a tensioning condition. The actuator 23 and tension member 27 are arranged to be configurable between two different states. In a first state, when the actuator 23 is released and there is no tension on the tension member 27, the force pulling the sections 24 together is relatively low so that they can articulate relative to one another. In effect, the introducer 2 is limp or floppy. In a second state when the actuator 23 is actuated to apply tension to the tension member 27, an axial force is applied along the introducer 2 tending to pull the nose portion 20 rearwardly towards its machine end 22. This also pulls the separate sections 24 of the introducer 2 against one another axially so that they effectively lock against one another at an orientation determined by the shape of the profiled end faces 26 of the sections. More particularly, the shape of the end faces 26 of the articulated sections 24 is selected so that, when the actuator 23 is tightened to lock the introducer 2 it will adopt a shape preferred for insertion of the tracheostomy tube assembly.

If the introducer 2 is provided separately from the tube 1, that is, it is not provided already inserted in the tube, the actuator 23 is initially set, in its first state, so that the introducer is untensioned and relatively limp. This enables the introducer 2 to be inserted into the tube 1 from its machine end 11, the introducer flexing as it moves along the curvature of the tube. Alternatively, the introducer 2 could be inserted in the tube while in a rigid state when outside the body. When the introducer 2 has been fully inserted, with the nose portion 21 projecting from the patient end 10 of the tube 1 and the actuator 23 lying against the machine end connector 16 the actuator is switched to the second state to apply tension to the tension member 27 and thereby lock the sections 24 together to form a rigid shaft. The rigid state of the introducer 2 supports the tube 1 and enables the assembly of the tube and introducer 2 to be inserted into the patient through the tracheostomy opening. When the assembly has been fully inserted, the actuator 23 is switched back to its relaxed, first state in which tension on the tension member 27 is reduced sufficiently to enable the sections 24 of the introducer 2 to articulate relative to one another. This in turn enables the introducer 2 to be pulled out of the tube freely, the introducer flexing to accommodate the shape of the tube 1 as it is removed and without significantly distorting the tube.

The introducer of the present invention is particularly useful with highly flexible tubes, such as tubes made with thin walls or with walls of a soft material and having a reinforcement to resist crushing. The assembly of the introducer with such a tube provides sufficient rigidity for insertion but, after removal of the introducer, enables the advantages of the flexible nature of the tube to be realised.

With reference now to FIGS. 3A and 3B there is shown an alternative introducer 302 according to the present invention, in two different states. FIG. 3A shows the introducer 302 in the first state, in its natural condition for insertion into or removal from a tracheostomy tube. FIG. 3B shows the introducer 302 in its second, tensioned state for use in a tracheostomy tube during insertion of the assembly of the tube and introducer into the body. The introducer 302 has an inner elongate component in the form of a shaft 303 formed by an inner articulated section 324 similar to in the introducer shown in FIG. 2 and an outer elongate component in the form of a sleeve 326 of a flexible material. A bore 325 extends along the length of the shaft 303. At its patient end, the shaft 303 has at its patient end 320 a nose portion 321 of the same shape as the introducer 2 shown in FIGS. 1 and 2. Externally, the shaft 303 is smooth and uninterrupted except for three annular gripping rings 328 of an elastomeric material extending around the outer sleeve 326. The outer sleeve 326 and the gripping rings 328 could both be moulded integrally of the same elastomeric material. The external diameter of the rings 328 is such that, in their natural state, it is just less than the internal diameter of the tube so that the introducer can be slid freely into or out of the tube. A tension member 327 extends along the bore 325 from the nose portion 321 to an actuator 323 at the machine end of the introducer. When the actuator 323 is adjusted to apply tension to the tension member 327 it acts to pull the nose portion 321 rearwardly slightly and shorten the overall length of the introducer 302. The construction of the inner articulated section 324 may be such as to allow a greater gap between adjacent sections so that the length of the articulated section can be reduced by a greater amount when a contracting tension is applied. Shortening the length of the introducer 302 causes the outer sleeve 326 to shorten and consequently causes the sleeve and its rings 328 to expand, as shown in FIG. 3B. The expansion is chosen such that the rings 327 contact and grip the inside of the tracheostomy tube towards its patient end. In this second state, when a force is applied by the clinician to the introducer 302, the internal grip of the introducer towards the patient end of the tube pulls it into the patient from the forward end. This reduces the risk that the tube may buckle or ride back along the introducer 302 during insertion. When the tube is correctly located in the patient the actuator 323 is released to allow the resilience of the outer sleeve 326 to return the introducer 302 to its natural, first state where it ceases to grip the inside of the tube and where the shaft 303 is relatively flexible. This enables the introducer 302 to be removed from the tube easily without disturbing it.

In the arrangement described with reference to FIGS. 3A and 3B it may not be essential to use an internal articulated section in the introducer but instead just the external resilient sleeve, providing that the tension member is stiff enough to enable sufficient pushing force to be applied during insertion of the assembly.

With reference now to FIGS. 4A and 4B there is shown a further alternative introducer 402 having an outer elongate component in the form of a sleeve 403 of a plastics material (although other materials, such as metal could be used) and an inner elongate component in the form of a stiff rod 404 slidable along a bore 405 along the sleeve. The outer sleeve 403 is shaped at its patient end 420 with a tapering nose portion 421 that is arranged, in use, to project from the patient end of a tracheostomy tube. The outer sleeve 403 has a region close to its patient end 420 with an externally-projecting gripping ring 407 encircling the sleeve. The sleeve could have more than one gripping ring. In its normal, relaxed, first state the external diameter of the gripping ring 407 is just less than the internal diameter of the tracheostomy tube so that the introducer 402 can be moved freely along the tracheostomy tube. Internally, the bore 405 of the sleeve 403 has an enlarged annular cavity 408 located just rearwardly of the gripping ring 407. The rod 404 extends from an actuator at its machine end to a patient end 425 located just rearwardly of the patient end of the bore 405 through the sleeve 403. Externally, the rod 404 has a smooth surface and an external diameter slightly less than the diameter of the bore 405 of the sleeve along most of its length. A short distance rearwardly of its patient end the rod 404 has a radially-enlarged barrel-shape projection 409 with tapering ends 410. In the normal, relaxed, first state of the introducer 402 the rod 404 is positioned in the bore 405 with its projection 409 located in the cavity 408 of the sleeve 403 as a relatively close, but not tight, fit. The diameter of the projection 409 on the rod 404 is greater than the diameter of the bore 405 along most of its length except in the cavity 408. The configuration of the actuator on the introducer 402 can be changed to displace the rod 404 forwardly, towards the patient end of the sleeve 403. In this respect the actuator can be of any of the forms mentioned above except that it functions in the opposite direction. Displacing the actuator to its gripping condition causes the rod 404 to move forwardly by about 10 mm, thereby moving the projection 409 on the rod forwardly out of the cavity 408 in the sleeve 403 to a position in line with the external gripping ring 407 on the sleeve. In this position, the diameter of the projection 409 on the rod 404 exceeds the normal diameter of the bore 405 so that the sleeve 403 is expanded outwardly, thereby enlarging the diameter of the gripping ring 407. This causes the gripping ring 407 to grip tightly the inside of the tracheostomy tube in the region of its patient end. In this condition the introducer 402 can be used to insert the tracheostomy tube through a tracheostomy opening by exerting a force at the patient end of the tube to pull the tube through the opening. Once the tube has been correctly inserted, the actuator is released to its normal position and the rod 404 is moved back to allow the gripping ring 407 to contract to its normal diameter and release its grip on the tube. This allows the introducer 402 to be removed from the tube. It will be appreciated that the introducer could be rearranged to grip the inside of the tube when the rod is pulled rearwardly, rather than when pushed forwardly.

The invention is not confined to use with tracheostomy tubes but could be used with other tubes for insertion in a body cavity.

1-14. (canceled)

15. A tube introducer adapted for insertion within the bore of a medico-surgical tube, characterised in that the introducer is selectively configurable from a first state where it is removable from the machine end of the tube to a second state where it supports the tube during insertion into the body.

16. A tube introducer according to claim 15, characterised in that the first state is less rigid than the second state.

17. A tube introducer according to claim 16, characterised in that the introducer includes a plurality of articulated sections that, in the first state, are only loosely retained with one another such that they can flex relative to one another and, in the second state, are retained more rigidly with one another.

18. A tube introducer according to claim 17, characterised in that the introducer includes a tension member extending along the introducer from its patient end to its machine end and an actuator at the machine end of the actuator arranged to apply tension to the tension member in the second state.

19. A tube introducer according to claim 15, characterised in that, in the second state, the introducer is expanded to grip the inside of the tube.

20. A tube introducer according to claim 15, characterised in that, in the second state, the introducer is expanded only in a region towards the patient end of the introducer.

21. A tube introducer according to claim 19, characterised in that the introducer has a plurality of gripping rings towards its patient end, and that the gripping rings are expanded in diameter in the second state.

22. A tube introducer according to claim 20, characterised in that the introducer has a plurality of gripping rings towards its patient end, and that the gripping rings are expanded in diameter in the second state.

23. A tube introducer according to claim 15, characterised in that the introducer includes two elongate components movable relative to one another from a first position where the introducer can be slid into and out of the tube to a second position where a part at least of the introducer grips the inside of the tube.

24. A tube introducer according to claim 15, characterised in that the introducer has a tapering patient end adapted to project from the patient end of the tube within which it is inserted such as to help guide insertion of the tube into the body.

25. A tube introducer adapted for insertion within the bore of a medico-surgical tube from the machine end of the tube, characterised in that the introducer has at least a region along its length towards its patient end with an external dimension that can be selectively changed from a first dimension where it enables the introducer to be slid into and out of the tube to a second, larger dimension where the outside of a part at least of the region can grip the inside of the tube to resist relative movement between the introducer and the tube.

26. A tube introducer according to claim 25, characterised in that the introducer includes two elongate components movable relative to one another from a first position where the introducer can be slid into and out of the tube to a second position where a part at least of the introducer grips the inside of the tube.

27. A tube introducer having a patient end adapted for insertion in the bore of a medico-surgical tube to extend substantially to the patient end of the tube and a machine end adapted to project externally beyond the machine end of the tube, characterised in that the introducer is arranged selectively to grip the inside of the tube close to the patient end of the tube such that the tube and introducer can be inserted into a body cavity by pushing the machine end of the introducer so as to pull the tube into the body cavity from its patient end.

28. A tube introducer according claim 27, characterised in that the introducer includes two elongate components movable relative to one another from a first position where the introducer can be slid into and out of the tube to a second position where a part at least of the introducer grips the inside of the tube.

29. A tube introducer according to claim 23, characterised in that the two elongate components include an outer sleeve and an inner elongate component that can be displaced longitudinally relative to the outer sleeve such that in the first position a part at least of the outer sleeve has a first diameter less than the internal diameter of the tube and in the second position the part of the outer sleeve has a larger diameter sufficient to grip the inside of the tube.

30. A tube introducer according to claim 26, characterised in that the two elongate components include an outer sleeve and an inner elongate component that can be displaced longitudinally relative to the outer sleeve such that in the first position a part at least of the outer sleeve has a first diameter less than the internal diameter of the tube and in the second position the part of the outer sleeve has a larger diameter sufficient to grip the inside of the tube.

31. A tube introducer according to claim 28, characterised in that the two elongate components include an outer sleeve and an inner elongate component that can be displaced longitudinally relative to the outer sleeve such that in the first position a part at least of the outer sleeve has a first diameter less than the internal diameter of the tube and in the second position the part of the outer sleeve has a larger diameter sufficient to grip the inside of the tube.

32. An assembly of a medico-surgical tube and a tube introducer adapted to be selectively configurable from a first state where it is removable from the machine end of the tube to a second state where it supports the tube during insertion into a body, wherein the introducer is inserted within the tube with the patient end of the introducer protruding at the patient end of the tube and the machine end of the introducer accessible at the machine end of the tube.

33. An assembly according to claim 32, characterised in that the tube is a tracheostomy tube.
